(19) United States
(12) Patent Application Publication
(10) Pub. No.: US 2016/0128924 A1
(43) Pub. Date: May 12, 2016

(54) Composition for Improving Skin Condition Containing a Vitamin Mixture

(71) Applicant: AMI Cosmetic Co., Ltd., Seoul (KR)

(72) Inventors: Kyung Rok Lee, Seoul (KR); Il Hong, Seoul (KR); Yu Jin Jeong, Gyeonggi-do (KR); Hyun Hwa Lee, Incheon (KR)

(21) Appl. No.: 14/887,985

(22) Filed: Oct. 20, 2015

(30) Foreign Application Priority Data
Nov. 11, 2014 (KR) 10-2014-0156386

(51) Int. Cl. A61K 8/60; A61K 8/49; A61K 8/33; A61Q 19/08; A61K 8/34; A61K 8/97; A61Q 19/00; A61K 8/42; A61K 8/55

(52) U.S. Cl. CPC A61K 8/60; A61K 8/42; A61K 8/4926; A61K 8/498; A61K 8/33; A61K 8/4953; A61K 8/55; A61K 8/49; A61K 8/4973; A61K 8/342; A61K 8/97; A61Q 19/00; A61Q 19/08; A61K 2800/592

(57) ABSTRACT

Disclosed is a composition for improving skin condition containing a vitamin mixture, and more specifically to a composition for improving skin condition containing a vitamin mixture including ten vitamins with excellent improved effect on skin condition. The composition for improving skin condition comprises a vitamin mixture of vitamin B5, vitamin B3, vitamin E, vitamin F, vitamin B9, vitamin B6, vitamin H, vitamin B12, vitamin C, and vitamin A.

### CROSS-REFERENCE TO RELATED APPLICATION

This application claims the benefit under 35 U.S.C. §119 of Korean Application Nos. 10-2014-0156386, filed Nov. 11, 2014, which is hereby incorporated by reference in its entirety.

### BACKGROUND

1. Field of the Invention

The present disclosure relates to a composition for improving skin condition containing a vitamin mixture, and more specifically to a composition for improving skin condition containing a vitamin mixture including ten vitamins with excellent improved effect on skin condition.

2. Discussion of Related Art

Skin aging decreases the secretion of various hormones internally regulating the metabolism, reduces the activity of immune cells and skin cells, and lowers biosynthesis of various proteins necessary for bodies. Thereby, intrinsic skin aging caused by internal factors is accelerated.

Also, the skin loses its elasticity and wrinkles increase, due to external factors, such as various pollutants or UV light. Thereby, extrinsic skin aging attributed to external factors is accelerated.

The skin is composed of the epidermis, the dermis, and the subcutaneous tissue. The dermis contains fiber components and matrix components. Collagen which takes up a large proportion of the dermis provides strength and elasticity to the skin and protects the skin. However, skin aging results in the reduction of the activity of fibroblasts which synthesize and secrete fiber components in the matrix of connective tissues and the number of cells. Thereby, the amount of fiber components (collagen) synthesized and moisture in skin cells are reduced, and the structure of stratum corneum is changed.

Also, the action of collagenases results in increasing crosslinks of collagen, which is the primary structural component of the skin, and degrading smoothness, hydration, and elasticity of the skin.

Recently, consumers are increasingly interested in anti-aging. Accordingly, consumers desire to purchase cosmetics for anti-aging and wrinkle improvement rises, and the demand for functional products rapidly increases.

### PATENT DOCUMENTS

(Patent Document 0001) Korean Patent Application No. 10-2012-0145799 (Dec. 13, 2012).

(Patent Document 0002) Korean Patent Application No. 10-2010-0138201 (Dec. 29, 2010).

### SUMMARY OF THE INVENTION

According to an embodiment of the present disclosure, there is provided a composition for improving skin condition comprising a vitamin mixture of vitamin B5, vitamin B3, vitamin E, vitamin F, vitamin B9, vitamin B6, vitamin H, vitamin B12, vitamin C, and vitamin A.

The vitamin mixture may include 100 parts by weight of vitamin B5, 90 to 110 parts by weight of vitamin B3, 1 to 3 parts by weight of vitamin E, 2 to 3 parts by weight of vitamin F, 0.05 to 0.15 parts by weight of vitamin B9, 0.05 to 0.15 parts by weight of vitamin B6, 0.05 to 0.15 parts by weight of vitamin H, 0.005 to 0.015 parts by weight of vitamin B12, 0.001 to 0.003 parts by weight of vitamin C, and 0.001 to 0.003 parts by weight of vitamin A.

The composition may include 100 parts by weight of a solvent, 20 to 30 parts by weight of the vitamin mixture, 20 to 50 parts by weight of a skin conditioner, 20 to 30 parts by weight of an extract of Hippophae rhamnoides fruit, 0.05 to 0.15 parts by weight of a thickener, and 50 to 70 parts by weight of an additive.

The effect of the present disclosure is not limited to the above effects, and is to include all effects that may be inferred from the features set forth in the detailed description or the claims.

### BRIEF DESCRIPTION OF THE DRAWINGS

FIG. 1 illustrates a change in skin radiance after applying a composition for improving skin condition according to an embodiment of the present disclosure;

FIG. 2 illustrates a result of skin-lifting after applying a composition for improving skin condition according to an embodiment of the present disclosure;

FIG. 3 illustrates a change in dark circles after applying a composition for improving skin condition according to an embodiment of the present disclosure; and

FIG. 4 illustrates a change in wrinkles around the eyes after applying a composition for improving skin condition according to an embodiment of the present disclosure.

### DETAILED DESCRIPTION OF EXEMPLARY EMBODIMENTS

Hereinafter, example embodiments of the present disclosure and physical properties of each ingredient will be described in detail. It should be understood, however, that they are provided only for explaining the present disclosure and do not limit the spirit and scope of the present disclosure.

According to an embodiment of the present disclosure, a composition for improving skin condition may include 100 parts by weight of a solvent, 20 to 30 parts by weight of the vitamin mixture, 20 to 50 parts by weight of a skin conditioner, 20 to 30 parts by weight of an extract of Hippophae rhamnoides fruit, 0.05 to 0.15 parts by weight of a thickener, and 50 to 70 parts by weight of an additive.

The vitamin mixture may include vitamin B5, vitamin B3, vitamin E, vitamin F, vitamin B9, vitamin B6, vitamin H, vitamin B12, vitamin C, and vitamin A. Particularly, the vitamin mixture may include 100 parts by weight of vitamin B5 (panthenol), 90 to 110 parts by weight of vitamin B3 (niacinamide), 1 to 3 parts by weight of vitamin E (tocopheryl acetate), 2 to 3 parts by weight of vitamin F (at least one selected from the group consisting of glyceryl linolenate and glyceryl linoleate), 0.05 to 0.15 parts by weight of vitamin B9 (folic acid), 0.05 to 0.15 parts by weight of vitamin B6 (pyridoxine HC1), 0.05 to 0.15 parts by weight of vitamin H (biotin), 0.005 to 0.015 parts by weight of vitamin B12 (cyanocobalamin), 0.001 to 0.003 parts by weight of vitamin C (sodium ascorbyl phosphate or ascorbic acid), and 0.001 to 0.003 parts by weight of vitamin A (retinyl palmitate).

The vitamin mixture has an excellent effect of improving skin radiance, skin hydration, face lifting, dark circles, and wrinkles around the eyes. When the content of the vitamin mixture is less than 20 parts by weight, the improvement in skin condition may be insufficient, and when the content is greater than 30 parts by weight, the cost efficiency may be reduced.

The solvent homogeneously mixes each ingredient of the composition for improving skin condition, and may contain purified water and ethanol at a ratio of 10:1 based on parts by weight. The mixed solvent may dissolve ingredients which are soluble in water and ingredients which are soluble in alcohol.

The skin conditioner may be contained in an amount of 20 to 50 parts by weight. For the skin conditioner, a conditioner, such as emollient, humectant, or occlusive agent, which is typically used for a composition for the skin may be used.

The extract of Hippophae rhamnoides fruit may be contained in an amount of 20 to 30 parts by weight, and has an excellent effect of improving skin radiance, skin hydration, face lifting, dark circles, and wrinkles around the eyes.

The Hippophae rhamnoides fruit may contain at least 300 mg of vitamin C, 3 to 4 mg of carotene, 10 to 15 mg of vitamin E, 0.2 to 0.4 mg of vitamin B1, 0.4 to 0.5 mg of vitamin B2, and 0.5 to 0.8 mg of folic acid. Dried seed may contain about 8% of an oily and fat ingredient. Seed oil may contain 2.1% of a non-alkaline substance.

The extract of Hippophae rhamnoides fruit, which contains a large amount of substances improving skin condition, may increase the effect of the skin conditioner in improving skin radiance, skin hydration, face lifting, dark circles, and wrinkles around the eyes.

When the content of the extract of Hippophae rhamnoides fruit is less than 20 parts by weight, the improvement in skin condition may be insufficient, and when the content is greater than 30 parts by weight, the cost efficiency may be reduced.

The extract of Hippophae rhamnoides fruit may be formed by putting washed and ground Hippophae rhamnoides fruit and an extractant into an extraction vessel, and performing extraction at a proper temperature for a certain period of time.

The extract of Hippophae rhamnoides fruit may be obtained by using centrifugation after removing solids through a filter, such as a filter paper, and filtering a supernatant under reduced pressure.

Examples of the extractant may include, but are not limited to, water, alcohol, or a mixture thereof, preferably lower alcohol with 1 to 4 carbon numbers or a solvent selected from the mixed solvent thereof, and more preferably methanol.

The extractant may be used in an amount of 200 to 1000 parts by weight with respect to 100 parts by weight of Hippophae rhamnoides fruit, and may be used in an amount of 500 parts by weight, taking into account efficiency during extraction and concentration of effective ingredients after extraction.

Examples of an extraction method may include an immersing extraction method, a thermal extraction method, a refluxing and cooling extraction method, and an ultrasonic extraction method, and preferably the immersing extraction method, where the extraction may be carried out once to five times. The extraction temperature may be 20° C. to 30° C., and preferably 20° C. to 25° C. The extraction duration may be 5 to 10 days, and preferably 7 days.

The extract of Hippophae rhamnoides fruit may be formed by sequentially extracting n-hexane, ethyl acetate, and n-butanol, and provided in the form of fractions obtained from each stage. For a fraction solvent for obtaining the factions of the extract of Hippophae rhamnoides fruit, at least one selected from the group consisting of n-hexane, ethyl acetate, and n-butanol may be used. The fractionation may typically use a reparatory funnel. The fractions may be obtained from the extract after once to five times of fractionation, preferably three times, and may be concentrated under reduced pressure after fractionation.

The thickener may be contained in an amount of 0.05 to 0.15 parts by weight. The thickener may increase the viscosity of the composition to enhance skin coating properties of the composition.

The type of the thickener is not specifically limited, but at least one selected from the group consisting of sodium stearate, algin, and xanthan gum may be used for the thickener.

When the content of the thickener is less than 0.05 parts by weight, the increase in viscosity may be insufficient, and when the content is greater than 0.15 parts by weight, the viscosity of the composition may be excessively raised, and thus the skin feeling during use may deteriorate.

The additive may be contained in an amount of 50 to 70 parts by weight. The additive may include glycerin, butylene glycol, an emulsifier, cyclohexasiloxane, alcohol, PEG-100 stearate, sucrose laurate, alkyl glucoside, carbomer, a mixture of acrylate and alkyl acrylate crosspolymer, and Citrus aurantium bergamia fruit oil.

The additive provides color and fragrance to the composition for improving skin condition, and enhances preservability. When the content of the additive is less than 50 parts by weight, the effect of the additive may be achieved insufficiently, and when the content is greater than 70 parts by weight, the cost efficiency may be reduced.

Hereinafter, the effect of the composition for improving skin condition according to the present disclosure on skin radiance, skin hydration, face lifting, dark circles, and wrinkles around the eyes, will be described in detail.

TABLE 1

| | Content |
|---|---|
| Ingredients | (%) |
| Purified water | To 100 |
| Skin conditioner | 16.0000 |
| Extract of Hippophae rhamnoides fruit | 10.0000 |
| Glycerin | 8.0000 |
| Butylene Glycol | 6.0000 |
| Niacinamide (vitamin B₃) | 5.0000 |
| Panthenol (vitamin B₅) | 5.0000 |
| Emulsifier | 5.0000 |
| Cyclohexasiloxane | 2.0000 |
| C14 to 22 alcohol | 1.0000 |
| PEG-100 stearate | 0.5000 |
| Sucrose laurate | 0.5000 |
| C12 to 20 akyl glucoside | 0.3000 |
| Carbomer | 0.2000 |
| Acrylate/C10 to 30 alkyl acrylate crosspolymer | 0.2000 |
| Tocopheryl acetate (vitamin E) | 0.1000 |
| Citrus aurantium bergamia fruit oil (fragrant) | 0.0500 |
| Thickener | 0.0400 |
| Glyceryl linolenate (vitamin F) | 0.0200 |
| Glyceryl linoleate (vitamin F) | 0.1000 |
| Folic acid (vitamin B₉) | 0.0050 |
| Pyridoxine HCl (vitamin B₆) | 0.0050 |
| Biotin (vitamin H) | 0.0050 |
| Cyanocobalamin (vitamin B₁₂) | 0.0005 |
| Sodium ascorbyl phosphate (vitamin C) | 0.0001 |
| Retinyl palmitate (vitamin A) | 0.0001 |

### Example 1

A composition for improving skin condition according to the present disclosure was prepared by putting the ingredients shown in the above Table 1 into a mixer.

The composition for improving skin condition prepared by Example 1 was applied to 22 women, and the improvement in skin radiance, skin hydration, face lifting, dark circles, and wrinkles around the eyes was measured. The results are shown in Tables 2 to 6 below.

In order to measure improvement effect of skin radiance, skin hydration, face lifting, dark circles, and wrinkles around the eyes, among women aged 30 to 60, 22 test subjects were selected, who did not fall under the standards of exclusion for the test, who understood the purpose, contents, etc. of the test and volunteered for the test, who understood possible adverse reaction and signed consents, and who could be observed during the test period. Women were excluded from the test subjects, who suffered from eczematous skin diseases (eczema, atopic dermatitis, etc.), who suffered from infectious skin diseases, who had allergic diathesis or hypersensitive (to food, pollen, etc.), who were allergic to cosmetics, pharmaceutics, usual exposure to sunlight, who were pregnant or nursing, who had serious nutritional disorders, who were drug or alcohol addicts, who had spots, acnes, tattoos, scars, erythema, telangiectasia, or burns on the test area, which may affect the test, who participated in the same test since which three months has not passed, and who were considered not suitable for the test according to the determination of the principal investigator of the test.

(Skin radiance was measured by applying 3 g of the composition for improving skin condition containing ten vitamins on each face of 22 women, morning and night, twice a day, for 28 days, and measuring a change in radiance using a Skin Gloss meter on Day 10 and Day 28 days after application, to obtain mean values thereof.

Skin hydration was measured using a Corneometer; face lifting using a Moire technique measuring device; dark circles using a Mexameter MX18; and wrinkles around the eyes using PRIMOS High Resolution.

Skin radiance and skin hydration were expressed in arbitrary units; face lifting in R, angle units; dark circles in Melanin index and arbitrary units; and wrinkles around the eyes in Ra, μm units.)

TABLE 2
[Test on skin radiance]

| | | | | | | After | After |
|---|---|---|---|---|---|---|---|
| Test subjects | | | | | Before | use | use |
| | No. | Initial | Age | Gender | use | (Day 10) | (Day 28) |
| Ex- | 1 | K S H | 38 | female | 80.00 | 81.00 | 83.00 |
| am- | 2 | Y O S | 41 | female | 81.00 | 84.00 | 85.33 |
| ple | 3 | L Y K | 48 | female | 61.00 | 78.33 | 79.33 |
| 1 | 4 | B M J | 46 | female | 59.67 | 59.33 | 75.33 |
| | 5 | L Y O | 40 | female | 70.00 | 77.00 | 73.67 |
| | 6 | N H S | 41 | female | 74.67 | 77.00 | 76.00 |
| | 7 | M H J | 43 | female | 63.67 | 71.33 | 79.67 |
| | 8 | L K H | 51 | female | 84.67 | 85.33 | 84.00 |
| | 9 | J S Y | 46 | female | 78.67 | 82.00 | 76.67 |
| | 10 | J M A | 50 | female | 79.33 | 80.00 | 82.00 |
| | 11 | S S Y | 39 | female | 73.67 | 60.67 | 74.33 |
| | 12 | K J Y | 43 | female | 65.00 | 71.00 | 82.33 |
| | 13 | K M R | 51 | female | 75.67 | 75.00 | 76.67 |
| | 14 | K Y R | 38 | female | 70.67 | 65.00 | 75.00 |
| | 15 | L K A | 50 | female | 81.33 | 81.00 | 81.00 |
| | 16 | S Y J | 55 | female | 65.00 | 70.33 | 84.00 |
| | 17 | K S Y | 55 | female | 77.67 | 63.33 | 84.00 |
| | 18 | L J S | 51 | female | 80.00 | 75.67 | 86.00 |
| | 19 | S H S | 53 | female | 71.00 | 79.67 | 81.33 |
| | 20 | L K O | 55 | female | 70.00 | 69.33 | 79.67 |
| | 21 | H C A | 54 | female | 72.67 | 83.67 | 83.33 |
| | 22 | H B Y | 55 | female | 83.33 | 70.33 | 89.33 |

| | | | |
|---|---|---|---|
| Mean value | 73.58 | 74.56 | 80.55 |

As shown in Table 2 above, skin radiance increased by 1.34% and 9.47% 10 days and 4 weeks after application of the composition for improving skin condition prepared by Example 1, respectively.

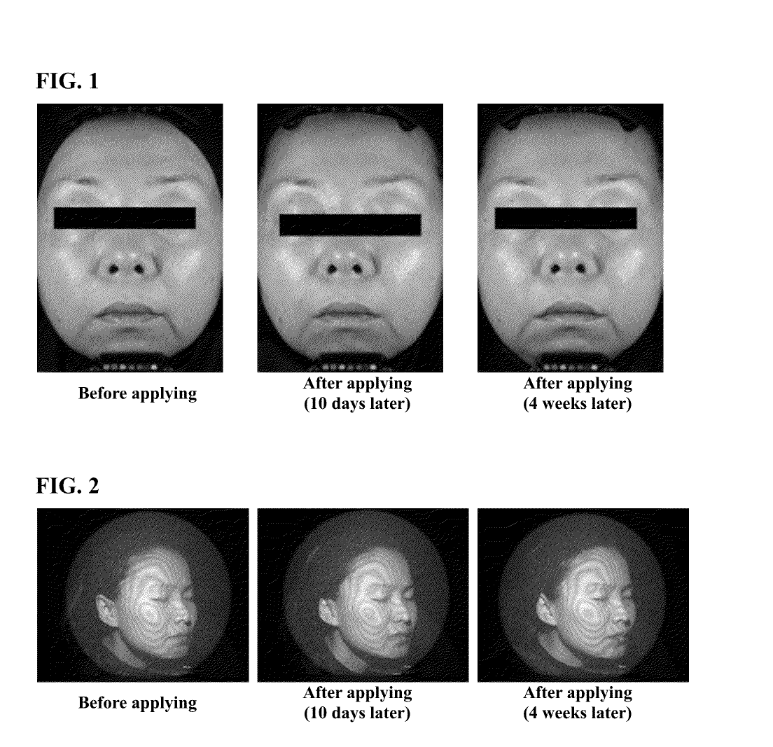

FIG. 1 is a photograph showing the skin condition of the test subject No. 21 who showed the most increased rate in skin radiance. As shown in FIG. 1, it can be understood that skin radiance of test subject No. 21 increased.

TABLE 3
[Test on skin hydration]

| | | | | | | After | After |
|---|---|---|---|---|---|---|---|
| Test subjects | | | | | Before | use | use |
| | No. | Initial | Age | Gender | use | (Day 10) | (Day 28) |
| Ex- | 1 | K S H | 38 | female | 52.00 | 56.37 | 62.90 |
| am- | 2 | Y O S | 41 | female | 68.30 | 72.77 | 78.70 |
| ple | 3 | L Y K | 48 | female | 57.17 | 61.03 | 64.73 |
| 1 | 4 | B M J | 46 | female | 48.03 | 57.93 | 56.00 |
| | 5 | L Y O | 40 | female | 53.10 | 55.57 | 61.90 |
| | 6 | N H S | 41 | female | 46.20 | 55.17 | 47.07 |
| | 7 | M H J | 43 | female | 37.87 | 43.43 | 42.97 |
| | 8 | L K H | 51 | female | 61.70 | 62.60 | 63.50 |
| | 9 | J S Y | 46 | female | 64.90 | 65.40 | 66.23 |
| | 10 | J M A | 50 | female | 34.07 | 53.80 | 54.17 |
| | 11 | S S Y | 39 | female | 68.67 | 70.43 | 70.27 |
| | 12 | K J Y | 43 | female | 57.47 | 70.27 | 61.47 |
| | 13 | K M R | 51 | female | 42.40 | 51.43 | 64.77 |
| | 14 | K Y R | 38 | female | 49.17 | 58.53 | 54.80 |
| | 15 | L K A | 50 | female | 66.87 | 65.23 | 63.30 |
| | 16 | S Y J | 55 | female | 59.87 | 66.07 | 65.47 |
| | 17 | K S Y | 55 | female | 68.07 | 70.67 | 75.57 |
| | 18 | L J S | 51 | female | 68.00 | 69.40 | 72.00 |
| | 19 | S H S | 53 | female | 57.33 | 60.53 | 72.07 |
| | 20 | L K O | 55 | female | 51.10 | 61.60 | 69.77 |
| | 21 | H C A | 54 | female | 73.90 | 74.80 | 58.37 |
| | 22 | H B Y | 55 | female | 56.90 | 67.03 | 64.13 |

| | | | |
|---|---|---|---|
| Mean value | 56.50 | 62.28 | 63.10 |

The composition for improving skin condition prepared by Example 1 was applied to the test subjects and the amount of moisture in stratum corneum was analyzed using the Corneometer. As a result, as shown in Table 3, the amount of moisture contained in the skin of the test subjects increased by 10.22% and 11.83% 10 days and 4 weeks after application, respectively.

TABLE 4
[Test on face lifting]

| | | | | | | After | After |
|---|---|---|---|---|---|---|---|
| Test subjects | | | | | Before | use | use |
| | No. | Initial | Age | Gender | use | (Day 10) | (Day 28) |
| Ex- | 1 | K S H | 38 | female | 29.06 | 28.69 | 30.47 |
| am- | 2 | Y O S | 41 | female | 45.65 | 44.30 | 43.76 |
| ple | 3 | L Y K | 48 | female | 51.21 | 50.65 | 49.40 |
| 1 | 4 | B M J | 46 | female | 38.66 | 37.37 | 36.71 |
| | 5 | L Y O | 40 | female | 36.53 | 36.31 | 34.70 |
| | 6 | N H S | 41 | female | 33.91 | 32.74 | 33.70 |
| | 7 | M H J | 43 | female | 28.77 | 27.96 | 27.13 |
| | 8 | L K H | 51 | female | 32.86 | 31.16 | 29.36 |
| | 9 | J S Y | 46 | female | 42.05 | 41.86 | 41.57 |
| | 10 | J M A | 50 | female | 42.62 | 42.46 | 41.86 |
| | 11 | S S Y | 39 | female | 43.46 | 51.67 | 36.87 |
| | 12 | K J Y | 43 | female | 37.03 | 36.67 | 36.89 |
| | 13 | K M R | 51 | female | 43.91 | 43.26 | 42.22 |
| | 14 | K Y R | 38 | female | 39.09 | 38.04 | 37.50 |
| | 15 | L K A | 50 | female | 45.61 | 44.43 | 43.72 |
| | 16 | S Y J | 55 | female | 47.29 | 46.12 | 44.37 |
| | 17 | K S Y | 55 | female | 46.97 | 45.00 | 44.34 |
| | 18 | L J S | 51 | female | 43.80 | 42.39 | 41.34 |
| | 19 | S H S | 53 | female | 43.69 | 42.04 | 41.55 |
| | 20 | L K O | 55 | female | 46.21 | 44.27 | 44.30 |
| | 21 | H C A | 54 | female | 48.96 | 48.44 | 46.19 |
| | 22 | H B Y | 55 | female | 46.24 | 44.37 | 43.56 |

| | | | |
|---|---|---|---|
| Mean value | 41.53 | 40.92 | 39.61 |

The composition for improving skin condition prepared by Example 1 was applied to the test subjects and the measured values of face lifting were analyzed using the Moire technique measuring device. As a result, as shown in Table 4, face lifting of the test subjects increased by 1.46% and 4.60% 10 days and 4 weeks after application, respectively, compared with before application.

FIG. 2 is a photograph showing the skin condition of test subject No. 11 who showed the most increased improvement effect on face lifting. As shown in FIG. 2 below, it can be understood that face lifting of test subject No. 11 significantly increased.

TABLE 5
[Test on dark circles]

| | | | | | | After | After |
|---|---|---|---|---|---|---|---|
| Test subjects | | | | | Before | use | use |
| | No. | Initial | Age | Gender | use | (Day 10) | (Day 28) |
| Ex- | 1 | K S H | 38 | female | 156.33 | 140.33 | 130.00 |
| am- | 2 | Y O S | 41 | female | 168.33 | 144.00 | 116.33 |
| ple | 3 | L Y K | 48 | female | 171.33 | 166.67 | 156.33 |
| 1 | 4 | B M J | 46 | female | 162.33 | 156.67 | 165.00 |
| | 5 | L Y O | 40 | female | 163.67 | 113.33 | 112.33 |
| | 6 | N H S | 41 | female | 125.67 | 113.33 | 112.33 |
| | 7 | M H J | 43 | female | 191.00 | 163.00 | 165.00 |
| | 8 | L K H | 51 | female | 137.00 | 137.00 | 125.00 |
| | 9 | J S Y | 46 | female | 162.67 | 163.33 | 168.67 |
| | 10 | J M A | 50 | female | 115.67 | 100.33 | 94.33 |
| | 11 | S S Y | 39 | female | 137.33 | 125.33 | 134.33 |
| | 12 | K J Y | 43 | female | 134.33 | 135.67 | 125.33 |
| | 13 | K M R | 51 | female | 181.67 | 165.67 | 158.00 |
| | 14 | K Y R | 38 | female | 162.67 | 156.33 | 150.33 |
| | 15 | L K A | 50 | female | 158.67 | 186.33 | 145.67 |
| | 16 | S Y J | 55 | female | 115.00 | 110.33 | 106.33 |
| | 17 | K S Y | 55 | female | 105.67 | 96.33 | 86.00 |
| | 18 | L J S | 51 | female | 101.67 | 96.00 | 83.00 |
| | 19 | S H S | 53 | female | 111.00 | 99.67 | 110.00 |
| | 20 | L K O | 55 | female | 131.00 | 121.67 | 109.33 |
| | 21 | H C A | 54 | female | 114.33 | 95.33 | 96.33 |
| | 22 | H B Y | 55 | female | 124.00 | 117.00 | 124.67 |

| | | | |
|---|---|---|---|
| Mean value | 142.33 | 133.94 | 127.73 |

The composition for improving skin condition prepared by Example 1 was applied to the test subjects and the measured values of dark circles were analyzed using the Mexameter MX18. As a result, as shown in Table 5, the dark circles of the test subjects decreased by 5.90% and 10.26% 10 days and 4 weeks after application, respectively, compared with before application.

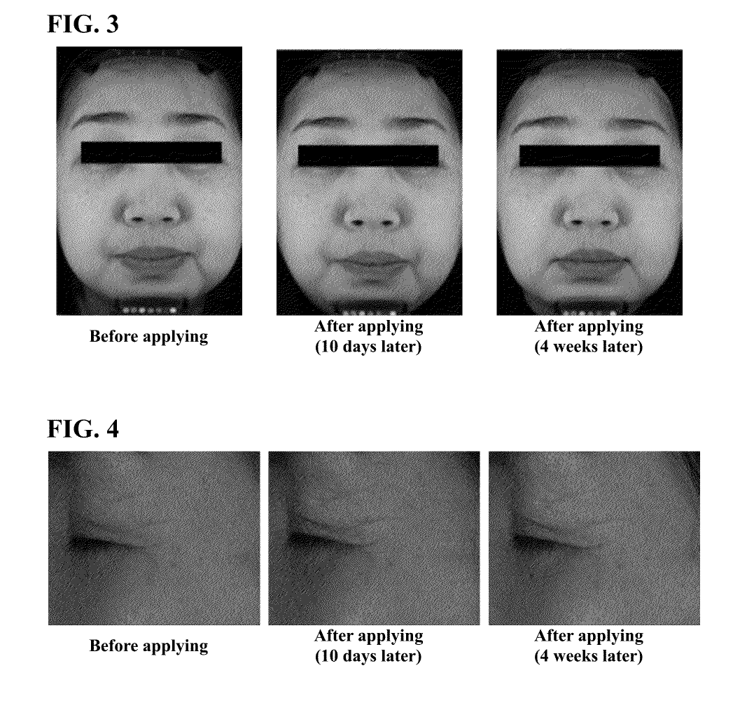

FIG. 3 is a photograph showing the skin condition of test subject No. 2 who showed the most increased improvement effect on dark circles. As shown in FIG. 3 below, it can be understood that dark circles of woman No. 2 significantly decreased.

TABLE 6
[Test on wrinkles around the eyes]

| | | | | | | After | After |
|---|---|---|---|---|---|---|---|
| Test subjects | | | | | Before | use | use |
| | No. | Initial | Age | Gender | use | (Day 10) | (Day 28) |
| Ex- | 1 | K S H | 38 | female | 21.70 | 19.00 | 19.73 |
| am- | 2 | Y O S | 41 | female | 15.13 | 17.07 | 15.63 |
| ple | 3 | L Y K | 48 | female | 14.27 | 13.93 | 13.80 |
| 1 | 4 | B M J | 46 | female | 20.80 | 20.27 | 19.70 |
| | 5 | L Y O | 40 | female | 16.47 | 14.77 | 13.50 |
| | 6 | N H S | 41 | female | 15.33 | 16.13 | 14.03 |
| | 7 | M H J | 43 | female | 14.97 | 15.60 | 15.57 |
| | 8 | L K H | 51 | female | 15.37 | 14.77 | 14.47 |
| | 9 | J S Y | 46 | female | 20.97 | 16.03 | 15.57 |
| | 10 | J M A | 50 | female | 14.70 | 15.27 | 13.30 |
| | 11 | S S Y | 39 | female | 10.67 | 12.77 | 12.27 |
| | 12 | K J Y | 43 | female | 15.80 | 16.00 | 13.77 |
| | 13 | K M R | 51 | female | 14.57 | 15.07 | 14.07 |
| | 14 | K Y R | 38 | female | 16.10 | 14.70 | 13.87 |
| | 15 | L K A | 50 | female | 20.67 | 20.30 | 22.90 |
| | 16 | S Y J | 55 | female | 31.85 | 23.73 | 23.23 |
| | 17 | K S Y | 55 | female | 17.17 | 15.87 | 16.55 |
| | 18 | L J S | 51 | female | 14.30 | 14.10 | 14.30 |
| | 19 | S H S | 53 | female | 27.90 | 25.33 | 27.86 |
| | 20 | L K O | 55 | female | 25.40 | 19.33 | 19.00 |
| | 21 | H C A | 54 | female | 13.63 | 13.23 | 16.23 |
| | 22 | H B Y | 55 | female | 16.17 | 16.93 | 16.97 |

| | | | |
|---|---|---|---|
| Mean value | 17.91 | 16.83 | 16.65 |

The composition for improving skin condition prepared by Example 1 was applied to the test subjects and the measured values of wrinkles around the eyes were analyzed using the PRIMOS High Resolution. As a result, as shown in Table 6, the wrinkles around the eyes of the test subjects were improved by 6.03% and 7.01% 10 days and 4 weeks after application, respectively, compared with before application.

FIG. 4 is a photograph showing the skin condition of woman No. 16 test subject No. 16 who showed the most increased improvement effect on wrinkles around the eyes. As shown in FIG. 4 below, it can be understood that wrinkles around the eyes of woman No. 16 significantly improved.

As demonstrated above, the composition for improving skin condition containing ten vitamins according to the present disclosure, which contains the vitamin mixture, is significantly effective on skin radiance, skin hydration, face lifting, dark circles, and wrinkles around the eyes.

Although the exemplary embodiments of the present disclosure have been disclosed for illustrative purposes, those skilled in the art will appreciate that various modifications, additions and substitutions are possible, without departing from the scope and spirit of the present disclosure as disclosed in the accompanying claims. Therefore, it should be understood that the forgoing description is by way of example only, and is not intended to limit the present disclosure. For example, each constituent explained in singular form may be carried out being dispersed, and likewise, constituents explained as being dispersed may be carried out in combined forms.

The scope of the present disclosure is defined by the foregoing claims, and it is intended that the present disclosure covers the modifications or variations of the present disclosure provided they come within the scope of the appended claims and their equivalents.

What is claimed is:

1. A composition for improving skin condition comprising a vitamin mixture consisting of vitamin B5, vitamin B3, vitamin E, vitamin F, vitamin B9, vitamin B6, vitamin H, vitamin B12, vitamin C, and vitamin A.

2. The composition of claim 1, wherein the vitamin mixture consists of 100 parts by weight of vitamin B5, 90 to 110 parts by weight of vitamin B3, 1 to 3 parts by weight of vitamin E, 2 to 3 parts by weight of vitamin F, 0.05 to 0.15 parts by weight of vitamin B9, 0.05 to 0.15 parts by weight of vitamin B6, 0.05 to 0.15 parts by weight of vitamin H, 0.005 to 0.015 parts by weight of vitamin B12, 0.001 to 0.003 parts by weight of vitamin C, and 0.001 to 0.003 parts by weight of vitamin A.

3. The composition of claim 1, which comprises 100 parts by weight of a solvent, 20 to 30 parts by weight of the vitamin mixture, 20 to 50 parts by weight of a skin conditioner, 20 to 30 parts by weight of an extract of Hippophae rhamnoides fruit, 0.05 to 0.15 parts by weight of a thickener, and 50 to 70 parts by weight of an additive.
